(19) United States
(12) Patent Application Publication
(10) Pub. No.: US 2005/0244872 A1
(43) Pub. Date: Nov. 3, 2005

(54) Breast cancer gene expression biomarkers

(75) Inventor: Cole Harris, Albuquerque, NM (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Exagen Diagnostics, Inc.

(21) Appl. No.: 11/112,944

(22) Filed: Apr. 22, 2005

Related U.S. Application Data
(60) Provisional application No. 60/564,757, filed on Apr. 23, 2004.

(51) Int. Cl. C12Q 1/68; C07H 21/04

(52) U.S. Cl. 435/6; 536/24.3

(57) ABSTRACT

The present invention provides compositions and their use in classifying breast tumors.

### CROSS REFERENCE

This application claims priority to U.S. Provisional Patent Application Ser. No. 60/564,757 filed Apr. 23, 2004, which is incorporated herein by reference in its entirety.

### INCORPORATION BY REFERENCE

A compact disc submission containing a Sequence Listing is hereby expressly incorporated by reference. The submission includes two compact discs (“COPY 1” and “COPY 2”), which are identical in content. Each disc contains the file entitled 05-325-US SeqListing.ST25.txt,” 82 KB in size, created Apr. 21, 2005.

### FIELD OF THE INVENTION

The invention relates generally to the fields of nucleic acids, nucleic acid detection, cancer, and breast cancer.

### BACKGROUND

Breast cancer is the most common cancer in women and the second most common cause of cancer death in the United States. While germ line mutations in BRCA1 or BRCA2 genes predispose women with the mutations to breast cancer, only about 5-10% of breast cancers are associated with these breast cancer susceptibility genes. Currently employed clinical indicators of breast cancer prognosis are not accurate in identifying patients likely to have a favorable outcome. As a result, many more patients are subjected to adjuvant chemotherapy than will benefit from such treatment (U.S. 20040058340 published Mar. 25, 2004).

Tumors not currently known to be associated with a germline mutation (“sporadic tumors”), constitute the majority of breast cancers (U.S. 20040058340). It is likely that non-genetic factors also play a significant role in the development of breast cancers. In any event, due to the increased morbidity and mortality if breast cancer is not detected early in its progression, considerable effort has been devoted to early detection of breast tumor development.

Breast cancer diagnosis typically requires histopathological proof of tumor presence. Histopathological examinations also provide information about prognosis and help guide selection of treatment regimens. Prognosis may also be established based upon clinical parameters such as tumor size, tumor grade, the age of the patient, and lymph node metastasis (U.S. 20040058340).

Accurate prognosis, or determination of distant metastasis-free survival, in breast cancer patients would permit selective administration of adjuvant chemotherapy, with women having poorer prognoses being given the most aggressive treatment.

The maturation of microarray technology has enabled the routine collection of genome-wide gene expression (RNA) data. In cancer diagnostics, several authors have shown that microarray data collected from tumors may be useful in differential diagnosis, tumor staging and prognosis. The data produced by these studies ideally represents a valuable resource for the development of new diagnostics.

Currently employed clinical indicators of breast cancer prognosis are not sufficiently accurate. As a result, many more patients are subjected to adjuvant chemotherapy than will benefit from such treatment. Thus, there remains a need in the art for better and more specific clinical predictors of breast cancer prognosis.

### SUMMARY OF THE INVENTION

The present invention provides compositions and their use in classifying breast tumors.

In one aspect, the present invention provides compositions comprising a breast cancer biomarker comprising or consisting of between 3 and 73 different probe sets, wherein at least 40% of the different probe sets comprise one or more isolated polynucleotides that selectively hybridize to a nucleic acid according to one of SEQ ID NO:1-29 or complements thereof; wherein the different probe sets in total selectively hybridize to at least three of the recited nucleic acids according to SEQ ID NO:1-29 or complements thereof.

In a second aspect, the present invention provides methods for classifying a breast tumor comprising:
(a) contacting a mRNA-derived nucleic acid sample obtained from a subject having a breast tumor with nucleic acid probes that, in total, selectively hybridize to three or more nucleic acid targets selected from the group consisting of SEQ ID NO:1-29 or complements thereof; wherein the contacting occurs under conditions to promote selective hybridization of the nucleic acid probes to the nucleic acid targets, or complements thereof, present in the nucleic acid sample;
(b) detecting formation of hybridization complexes between the nucleic acid probes to the nucleic acid targets, or complements thereof, wherein a number of such hybridization complexes provides a measure of gene expression of the one or more nucleic acids according to SEQ ID NO:1-29; and
(c) correlating an alteration in gene expression of the one or more nucleic acids according to SEQ ID NO:1-29 relative to control with a a breast cancer classification.

### DETAILED DESCRIPTION OF THE INVENTION

All references cited are herein incorporated by reference in their entirety.

Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

The present invention provides novel compositions and methods for their use in classifying breast tumors. As used herein, the term “classifying” means to determine one or more features of the breast tumor or the prognosis of a patient from whom a breast tissue sample is taken, including the following:
(a) Diagnosis of breast cancer (benign vs. malignant tumor);
(b) Metastatic potential, potential to metastasize to specific organs, risk of recurrence, or course of the tumor;
(c) Stage of the tumor;
(d) Patient prognosis in the absence of chemotherapy or hormonal therapy;
(e) Prognosis of patient response to treatment (chemotherapy, radiation therapy, and/or surgery to excise tumor)
(f) Predicted optimal course of treatment for the patient;
(g) Prognosis for patient relapse after treatment; and
(h) Patient life expectancy.

In a first aspect, the present invention provides compositions comprising or consisting of a breast cancer biomarker comprising or consisting of between 3 and 73 different probe sets, wherein at least 40% of the different probe sets comprise or consist of one or more isolated polynucleotides that selectively hybridize to a nucleic acid according to one of SEQ ID NO:1-29 or their complements; wherein the different probe sets in total selectively hybridize to at least three of the recited nucleic acids according to SEQ ID NO:1-29 or their complements.

While results obtained using two of the markers disclosed herein to classify a breast tumor are statistically significant, the inventors believe that the clinical diagnostic utility of further subsets of these markers are greater than the clinical diagnostic utility of pairs of markers. Such combinations consisting of more than two probes may better characterize the complexity of gene expression abnormalities with particular phenotypes in breast cancer. Thus, in various preferred embodiments of the first aspect of the invention, the composition comprises a breast cancer biomarker comprising or consisting of 3, 4, 5, 6, 7, 8, 9, 10, 11, 12, 13, 14, 15, 16, 17, 18, 19, 20, 21, 22, 23, 24, 25, 26, 27, 28, or 29 different probe sets that selectively hybridize to a nucleic acid according to one of SEQ ID NO:1-29 or their complements, wherein the different probe sets in total selectively hybridize to at least 3, 4, 5, 6, 7, 8, 9, 10, 11, 12, 13, 14, 15, 16, 17, 18, 19, 20, 21, 22, 23, 24, 25, 26, 27, 28, or 29 of the recited nucleic acids according to SEQ ID NO:1-29 or their complements. In each of these embodiments, it is further preferred that at least 45%, 50%, 55%, 60%, 65%, 70%, 80%, 85%, 90%, 95%, or 100% of the probe sets for a given breast cancer biomarker comprise or consist of one or more isolated polynucleotides that selectively hybridize to a nucleic acid according to SEQ ID NO:1-29, or their complements. As will be apparent to those of skill in the art, as the percentage of probe sets that comprise or consist of one or more isolated polynucleotides that selectively hybridize to a nucleic acid according to SEQ ID NO:1-29, or their complements, the maximum number of probe sets in the breast cancer biomarker will decrease accordingly. Thus, for example, where at least 80% of the probe sets comprise or consist of one or more isolated polynucleotides that selectively hybridize to a nucleic acid according to SEQ ID NO:1-29, or their complements, the breast cancer marker will consist of between 3 and 36 probe sets. Those of skill in the art will recognize the various other permutations encompassed by the compositions according to the various embodiments of the third aspect of the invention.

The compositions of the present invention are useful, for example, in classifying human breast tissue from a mammalian, preferably a human, subject. The compositions can be used, for example, to determine the expression levels in tissue of mRNA complementary to the recited genes. The compositions of this first aspect of the invention are especially preferred for use in RNA expression analysis from the genes in a tissue of interest, such as breast tissue samples (including but not limited to biopsies, lumpectomy samples, and solid tumor samples), fibroids, circulating tumor cells that have been shed from a tumor, blood samples (such as blood smears), and bone marrow cells. Such polynucleotides according to this aspect of the invention can be of any length that permits selective hybridization to the nucleic acid of interest. In various preferred embodiments of this aspect of the invention and related aspects and embodiments disclosed below, the isolated polynucleotides comprise or consist of at least 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 nucleotides according to a nucleic acid selected from the group consisting of SEQ ID NO:1-29, or their complements. In further embodiments, an isolated polynucleotide according to this first aspect of the invention comprise or consist of a nucleic acid according to one of SEQ ID NO:1-29, or their complements.

The term “polynucleotide” as used herein refers to DNA or RNA, preferably DNA, in either single- or double-stranded form, wherein the polynucleotides must comprise a sequence complementary to deposited genes. In a preferred embodiment, the polynucleotides are single stranded nucleic acids that are “anti-sense” to the recited nucleic acid (or its corresponding RNA sequence). The term “polynucleotide” encompasses nucleic acids containing known analogues of natural nucleotides which have similar or improved binding properties, for the purposes desired, as the reference polynucleotide. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs), methylphosphonate linkages or alternating methylphosphonate and phosphodiester linkages (Strauss-Soukup (1997) Biochemistry 36:8692-8698), and benzylphosphonate linkages, as discussed in U.S. Pat. No. 6,664,057; see also Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press).

An “isolated” polynucleotide as used herein for all of the aspects and embodiments of the invention is one which is free of sequences which naturally flank the polynucleotide in the genomic DNA of the organism from which the nucleic acid is derived, and preferably free from linker sequences found in nucleic acid libraries, such as cDNA libraries. Moreover, an “isolated” polynucleotide is substantially free of other cellular material, gel materials, and culture medium when produced by recombinant techniques, or substantially free of chemical precursors or other chemicals when chemically synthesized. The polynucleotides of the invention may be isolated from a variety of sources, such as by PCR amplification from genomic DNA, mRNA, or cDNA libraries derived from mRNA, using standard techniques; or they may be synthesized in vitro, by methods well known to those of skill in the art, as discussed in U.S. Pat. No. 6,664,057 and references disclosed therein. Synthetic polynucleotides can be prepared by a variety of solution or solid phase methods. Detailed descriptions of the procedures for solid phase synthesis of polynucleotide by phosphite-triester, phosphotriester, and H-phosphonate chemistries are widely available. (See, for example, U.S. Pat. No. 6,664,057 and references disclosed therein). Methods to purify polynucleotides include native acrylamide gel electrophoresis, and anion-exchange HPLC, as described in Pearson (1983) J. Chrom. 255:137-149. The sequence of the synthetic polynucleotides can be verified using standard methods.

As used herein with respect to all aspects and embodiments of the invention, a “probe set” refer to a group of one or more isolated polynucleotides that each selectively hybridize to the same target (for example, a specific mRNA) that can be used, for example, in breast cancer classification. Thus, a single “probe set” may comprise any number of different isolated polynucleotides that selectively hybridize to a given target. For example, a probe set that selectively hybridizes to SEQ ID NO:10 may comprise probes for a single 100 nucleotide segment of SEQ ID NO:10, or for a 100 nucleotide segment of SEQ ID NO:10 and also a different 100 nucleotide segment of SEQ ID NO:10, or both these in addition to a separate 10 nucleotide segment of SEQ ID NO:10, or 500 different 10 nucleotide segments of SEQ ID NO:10 (such as, for example, fragmenting a larger probe into many individual short polynucleotides). Those of skill in the art will understand that many such permutations are possible.

The compositions of the invention can be in lyophilized form, or preferably comprise a solution containing the at different probe sets. Such a solution can be made as such, or the composition can be prepared at the time of hybridizing the polynucleotides to a target sequence, as discussed below. Alternatively, the compositions can be placed on a solid support, such as in a microarray or microplate format.

In all of the above embodiments, it is further preferred that the polynucleotides are labeled with a detectable label. In a preferred embodiment, the detectable labels on the different polynucleotides of the nucleic acid composition are distinguishable from each other, for example, to facilitate differential determination of their signals when conducting hybridization reactions using multiple polynucleotides. Methods for detecting the label include, but are not limited to spectroscopic, photochemical, biochemical, immunochemical, physical or chemical techniques. For example, useful labels include but are not limited to radioactive labels such as ³²P, ³H, and ¹⁴C; fluorescent dyes such as fluorescein isothiocyanate (FITC), rhodamine, lanthanide phosphors, and Texas red, ALEXIS™ (Abbott Labs), CY™ dyes (Amersham); electron-dense reagents such as gold; enzymes such as horseradish peroxidase, beta-galactosidase, luciferase, and alkaline phosphatase; calorimetric labels such as colloidal gold; magnetic labels such as those sold under the mark DYNABEADS™; biotin; dioxigenin; or haptens and proteins for which antisera or monoclonal antibodies are available. The label can be directly incorporated into the polynucleotide, or it can be attached to a probe or antibody which hybridizes or binds to the polynucleotide. The labels may be coupled to the probes by any means known to those of skill in the art. In a various embodiments, the polynucleotides are labeled using nick translation, PCR, or random primer extension (see, e.g., Sambrook et al. supra).

As discussed above, the inventors have identified optimal markers of altered RNA expression associated with breast cancer. Thus, in a second aspect, the invention provides methods for classifying a breast tumor comprising:
(a) contacting a mRNA-derived nucleic acid sample obtained from a subject having a breast tumor with nucleic acid probes that, in total, selectively hybridize to two or more nucleic acid targets selected from the group consisting of SEQ ID NO:1-29 or complements thereof; wherein the contacting occurs under conditions to promote selective hybridization of the nucleic acid probes to the nucleic acid targets, or complements thereof, present in the nucleic acid sample;
(b) detecting formation of hybridization complexes between the nucleic acid probes to the nucleic acid targets, or complements thereof, wherein a number of such hybridization complexes provides a measure of gene expression of the one or more nucleic acids according to SEQ ID NO:1-29; and
(c) correlating an alteration in gene expression (ie, an increase or decrease) of the one or more nucleic acids according to SEQ ID NO:1-29 relative to control with a breast cancer classification. In a preferred embodiment, the classification comprises breast cancer recurrence.

The methods according to the second aspect of the invention detect alterations in gene expression of one or more of the markers according to SEQ ID NO:1-29 relative to a control with a modification in expression relative to control correlating with a classification of the breast tumor as likely to recur.

Any control known in the art can be used in the methods of the invention. For example, the expression level of a gene known to be expressed at a relatively constant level in both cancerous and non-cancerous tumor tissue can be used for comparison. Alternatively, the expression level of the genes targeted by the probes can be analyzed in non-cancerous RNA samples equivalent to the test sample. Those of skill in the art will recognize that many such controls can be used in the methods of the invention.

In the second aspect of the invention the methods are used to detect gene expression alterations associated with breast cancer. As used herein “associated with breast cancer” means that an altered expression level of one or more of the markers can be used to classify a feature of the breast tumor or the prognosis of a patient from whom the nucleic acid sample was taken, including the following:
(a) Diagnosis of breast cancer (benign vs. malignant tumor);
(b) Metastatic potential, potential to metastasize to specific organs, or course of the tumor;
(c) Stage of the tumor;
(d) Patient prognosis in the absence of chemotherapy or hormonal therapy;
(e) Prognosis of patient response to treatment (chemotherapy, radiation therapy, and/or surgery to excise tumor)
(f) Predicted optimal course of treatment for the patient;
(g) Prognosis for patient relapse after treatment; and
(h) Patient life expectancy.

Thus, the methods of this aspect of the invention provide information on, for example, breast cancer diagnosis, and patient prognosis in the presence or absence of chemotherapy, a predicted optimal course for treatment of the patient, and patient life expectancy. In a preferred embodiment, the breast cancer classification comprises a prognosis of the recurrence of the breast tumor. In a further preferred embodiment, an altered expression level of the one or more nucleic acid targets is correlated with an increased recurrence rate of the breast tumor compared to control. As used herein, “recurrence” means tumor return at the same site, metastasis or death from breast cancer.

In a further preferred embodiment, alterations in the normal expression levels of the one or more nucleic acid targets are correlated with a higher risk of recurrence of the breast tumor. One skilled in the art will understand that “alteration in the expression levels” means any deviation from the level of expression relative to the same normal healthy tissue. It is further understood that “increased risk” means to be at a higher risk relative to all others having similar or identical clinical and/or pathological characteristics, in the absence of the information obtained using the markers as described herein.

As used herein for all aspects and embodiments of the method, an alteration (ie: an increase or decrease) in gene expression relative to control is any increase or decrease relative to control, such as a normal tissue counterpart of the disease state or other appropriate control. In various embodiments, the increase or decrease is at least a 10%, 20%, 30%, 40%, 50%, 60%, 70%, 80%, 90%, 100%, 200%, or greater increase or decrease.

Thus, the invention further provides methods for making a treatment decision for a breast cancer patient, comprising carrying out the methods for classifying a breast tumor according to the second aspect of the invention, and embodiments thereof, and then weighing the results in light of other known clinical and pathological risk factors, in determining a course of treatment for the breast cancer patient. For example, a patient that is shown by the methods of the invention to have an increased risk of recurrence could be treated more aggressively with standard therapies, such as chemotherapy, radiation therapy, and/or surgical removal of the tumor.

The RNA sample used in the methods of the present invention can be from any source useful in classifying a breast tumor, including but not limited to breast tissue samples (including but not limited to biopsies, lumpectomy samples, and solid tumor samples), fibroids, circulating tumor cells that have been shed from a tumor, and blood samples (such as blood smears), and bone marrow cells. In a preferred embodiment, the RNA sample is a human RNA sample. It will be understood by those of skill in the art that the RNA sample does not require isolation of RNA, as a complex sample mixture containing RNA to be tested can be used, such as a cell or tissue sample analyzed by in situ hybridization.

In a most preferred embodiment, the probe comprises single stranded anti-sense polynucleotides of the nucleic acid compositions of the invention. For example, in mRNA fluorescence in situ hybridization (FISH) (ie. FISH to detect messenger RNA), only an anti-sense probe strand hybridizes to the single stranded mRNA in the RNA sample, and in that embodiment, the “sense” strand oligonucleotide can be used as a negative control.

Alternatively, DNA probes can be used as probes. In this embodiment, it is preferable to distinguish between hybridization to cytoplasmic RNA and hybridization to nuclear DNA. There are two major criteria for making this distinction: (1) copy number differences between the types of targets (hundreds to thousands of copies of RNA vs. two copies of DNA) which will normally create significant differences in signal intensities and (2) clear morphological distinction between the cytoplasm (where hybridization to RNA targets would occur) and the nucleus will make signal location unambiguous. Thus, when using double stranded DNA probes, it is preferred that the method further comprises distinguishing the cytoplasm and nucleus in cells being analyzed within the bodily fluid sample. Such distinguishing can be accomplished by any means known in the art, such as by using a nuclear stain such as Hoeschst 33342, or DAPI which delineate the nuclear DNA in the cells being analyzed. In this embodiment, it is preferred that the nuclear stain is distinguishable from the detectable probe. It is further preferred that the nuclear membrane be maintained, i.e that all the Hoeschet or DAPI stain be maintained in the visible structure of the nucleus.

Any conditions in which the probe binds selectively to the RNA sample to form a hybridization complex, and minimally or not at all to other sequences, can be used in the methods of the present invention. The exact conditions used will depend on the length of the polynucleotides probes employed, their GC content, as well as various other factors as is well known to those of skill in the art. (See, for example, Tijssen (1993) Laboratory Techniques in Biochemistry and Molecular Biology—Hybridization with Nucleic Acid Probes part I, chapt 2, “Overview of principles of hybridization and the strategy of nucleic acid probe assays,” Elsevier, N.Y. (“Tijssen”)). In one embodiment, stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent wash conditions is a 0.2×SSC wash at 65° C. for 15 minutes (see, e.g., Sambrook (1989) Molecular Cloning: A Laboratory Manual (2nd ed.) Vol. 1-3, Cold Spring Harbor Laboratory, Cold Spring Harbor Press, NY (“Sambrook”) for a description of SSC buffer). Often, a high stringency wash is preceded by a low stringency wash to remove background probe signal.

In a preferred embodiment of hybridization and wash conditions, the methods comprise contacting the RNA sample with the probe under stringent hybridization conditions, detecting the formation of hybridization complexes, and quantifying the RNA expression level of the disclosed genes (from the probe) in the RNA sample. A variety of methods for specific nucleic acid measurement using nucleic acid hybridization techniques are known to those of skill in the art. See. e.g., NUCLEIC ACID HYBRIDIZATION, A PRACTICAL APPROACH, Ed. Hames, B. D. and Higgins, S. J., IRL Press, 1985; Sambrook.

Any method for evaluating the presence or absence of target RNA in a sample can be used, such as by Northern blotting methods, in situ hybridization, polymerase chain reaction (PCR) analysis, or array based methods.

In a preferred embodiment, detection is performed by in situ hybridization (“ISH”). In situ hybridization assays are well known to those of skill in the art. Generally, in situ hybridization comprises the following major steps (see, for example, U.S. Pat. No. 6,664,057): (1) fixation of tissue, biological structure, or nucleic acid sample to be analyzed; (2) pre-hybridization treatment of the tissue, biological structure, or nucleic acid sample to increase accessibility of the nucleic acid sample (within the tissue or biological structure in those embodiments), and to reduce nonspecific binding; (3) hybridization of the probe to the nucleic acid sample; (4) post-hybridization washes to remove probe not bound in the hybridization and (5) detection of the hybridized nucleic acid fragments. The reagent used in each of these steps and their conditions for use varies depending on the particular application. In a particularly preferred embodiment, ISH is conducted according to methods disclosed in U.S. Pat. Nos. 5,750,340 and/or 6,022,689, incorporated by reference herein in their entirety.

In a typical in situ hybridization assay, cells are fixed to a solid support, typically a glass slide. The cells are typically denatured with heat or alkali and then contacted with a hybridization solution to permit annealing of labeled probes specific to the nucleic acid sequence encoding the protein. The polynucleotides of the invention are typically labeled, as discussed above. In some applications it is necessary to block the hybridization capacity of repetitive sequences. In this case, human genomic DNA or Cot-1 DNA is used to block non-specific hybridization.

In a further embodiment, an array-based format can be used in which the polynucleotides of the invention can be arrayed on a surface and the RNA sample is hybridized to the polynucleotides on the surface. In this type of format, large number of different hybridization reactions can be run essentially “in parallel.” This provides rapid, essentially simultaneous, evaluation of a large number of genes. Methods of performing hybridization reactions in array based formats are also described in, for example, Pastinen (1997) Genome Res. 7:606-614; (1997) Jackson (1996) Nature Biotechnology 14:1685; Chee (1995) Science 274:610; WO 96/17958. Methods for immobilizing the polynucleotides on the surface and derivatizing the surface are known in the art; see, for example, U.S. Pat. No. 6,664,057.

In each of the above aspects and embodiments, detection of hybridization is typically accomplished through the use of a detectable label on the polynucleotides of the invention, such as those described above; in some alternatives, the label can be on the target nucleic acids. The label can be directly incorporated into the polynucleotide, or it can be attached to a probe or antibody which hybridizes or binds to the polynucleotide. The labels may be coupled to the probes in a variety of means known to those of skill in the art, as described above. In a preferred embodiment, the detectable labels on the different polynucleotides of the nucleic acid composition are distinguishable from each other. The label can be detectable can be by any techniques, including but not limited to spectroscopic, photochemical, biochemical, immunochemical, physical or chemical techniques, as discussed above.

In a further aspect, the present invention provides kits for use in the methods of the invention, comprising the compositions of the invention and instructions for their use. In a preferred embodiment, the polynucleotides are labeled, most preferably where the labels on each polynucleotide in a given probe set are the same, and differ from the detectable labels on the polynucleotides in other probe sets are different and distinguishable, as disclosed above. In a further preferred embodiment, the probes are provided in solution, most preferably in a hybridization buffer to be used in the methods of the invention. In further embodiments, the kit also comprises wash solutions and/or pre-hybridization solutions.

### EXAMPLE 1

Currently Employed Clinical Indicators of Breast Cancer Prognosis are not Accurate in Identifying Patients Likely to Have a Favorable Outcome.

As a result, many more patients are subjected to adjuvant chemotherapy than will benefit from such treatment. Van't Veer et al (2002) addressed the question of identifying a gene expression profile correlating with prognosis. The data collected by his group consisted of gene expression measurements across 24481 genes for 97 breast tumor samples with accompanying clinical data. Applying a univariate gene selection mechanism, they identified a group of 70 genes useful in predicting prognosis:

Van't Veer 70 gene marker

| | |
|---|---|
| Accuracy | 80.8% |
| Sensitivity | 91.2% |
| Specificity | 72.7% |

However, the clinical utility of a 70 gene marker is limited by the cost and complexity of coordinating 70 measurements.

The Van't Veer dataset was partitioned by the original investigators into a training dataset consisting of data collected from 44 good prognosis patient samples and 34 poor prognosis patient samples, and a test dataset consisting of data collected from 7 good prognosis patient samples and 12 poor prognosis patient samples. The training portion of the data was used by the authors to identify their 70 gene marker, and the test portion of the data to independently test the performance of this marker.

We used the training subset of the data to develop an ensemble of 8512 five-gene biomarkers and 2624 3-gene biomarkers. A variant of linear discriminant analysis was used to define the relationship between the gene expression values in each biomarker in the training phase of the analysis. In this step, the marker sets are identified by their ability to categorize the training samples into good or poor prognosis groups. However other methods could be used to define this relationship. The performance of each biomarker was evaluated according to its accuracy in predicting prognosis. As used herein, accuracy refers to the proportion of samples correctly identified as having good or poor prognosis. In the training data, a technique known as leave-one-out-cross-validation (loocv) was used to estimate the accuracy.

We have identified a set of 29 genes that, when used as biomarkers in combinations of two or more genes from the set, biomarker expression patterns correlate with breast cancer prognosis with respect to disease free survival. The cDNA sequence for each of these sequences is presented in SEQ ID NOS:1-29.

For example, use of three gene biomarkers was comparable in accuracy to the original investigators' 70-gene solution. Extending the analysis to 5-gene biomarkers produced significantly more accurate markers:

| | Accuracy | Sensitivity | Specificity |
|---|---|---|---|
| Van't Veer | 80.8% | 91.2% | 72.7% |
| 70 gene | | | |
| Herein | 88.5% | 94.1% | 84.1% |
| 5 gene | | | |

Accuracy is defined above. Sensitivity refers to the proportion of poor prognosis samples correctly classified as such, and specificity refers to the proportion of good prognosis samples correctly classified as such.

Additionally, this particular five gene marker correctly classified 18 of the 19 independent test samples. This is a very encouraging result, and demonstrates the prognostic information contained in gene expression data.

Table 1 provides examples of test accuracy on the training and test data using 5 marker sets:

TABLE 1

| Biomarker, | | | | |
|---|---|---|---|---|
| test data | | | | |
| accuracy, | | HUGO | | |
| training data | | gene | | Accession |
| accuracy, | Analyte | symbol | HUGO gene description | number |
| BC1 | 1 | | Homo sapiens mRNA; | AL080059 |
| 94.7% | | | cDNA DKFZp564H142 | (SEQ ID: 1) |
| 85.9% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | RAB27B | RAB27B, member RAS | NM_004163 |
| | | | oncogene family | (SEQ ID NO: 3) |
| | 4 | GCN1L1 | GCN1 (general control of | N38891 |
| | | | amino-acid synthesis 1, | (SEQ ID NO: 4) |
| | | | yeast)-like 1 | |
| | 5 | EXT1 | exostoses (multiple) 1 | NM_000127 |
| | | | | (SEQ ID NO: 5) |
| BC2 | 1 | | Homo sapiens mRNA; | AL080059 |
| 94.7% | | | cDNA DKFZp564H142 | (SEQ ID: 1) |
| 88.5% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | GCN1L1 | GCN1 (general control of | N38891 |
| | | | amino-acid synthesis 1, | (SEQ ID NO: 4) |
| | | | yeast)-like 1 | |
| | 4 | KIAA1104 | KIAA1104 protein | NM_014968 |
| | | | | (SEQ ID NO: 6) |
| | 5 | EXT1 | exostoses (multiple) 1 | NM_000127 |
| | | | | (SEQ ID NO: 5) |
| BC3 | 1 | | Homo sapiens mRNA; | AL080059 |
| 89.5% | | | cDNA DKFZp564H142 | (SEQ ID: 1) |
| 89.7% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | GCN1L1 | GCN1 (general control of | N38891 |
| | | | amino-acid synthesis 1, | (SEQ ID NO: 4) |
| | | | yeast)-like 1 | |
| | 4 | MP1 | metalloprotease 1 (pitrilysin | NM_014889 |
| | | | family) | (SEQ ID NO: 7) |
| | 5 | EXT1 | exostoses (multiple) 1 | NM_000127 |
| | | | | (SEQ ID NO: 5) |
| BC4 | 1 | | Homo sapiens mRNA; | AL080059 |
| 78.9% | | | cDNA DKFZp564H142 | (SEQ ID: 1) |
| 88.5% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | ALDH4A1 | aldehyde dehydrogenase 4 | NM_003748 |
| | | | (glutamate gamma- | (SEQ ID NO: 8) |
| | | | semialdehyde | |
| | | | dehydrogenase; pyrroline-5- | |
| | | | carboxylate dehydrogenase) | |
| | 4 | | ESTs | AW014921 |
| | | | | (SEQ ID NO: 9) |
| | 5 | | Homo sapiens cDNA: | AK026372 |
| | | | FLJ22719 fis, clone | (SEQ ID NO: 10) |
| | | | HSI14307 | |
| BC5 | 1 | | Homo sapiens mRNA; | AL080059 |
| 89.5% | | | cDNA DKFZp564H142 | (SEQ ID: 1) |
| 85.9% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | | ESTs | AL310524 |
| | | | | (SEQ ID NO: 11) |
| | 4 | GCN1L1 | GCN1 (general control of | N38891 |
| | | | amino-acid synthesis 1, | (SEQ ID NO: 4) |
| | | | yeast)-like 1 | |
| | 5 | EXT1 | exostoses (multiple) 1 | NM_000127 |
| | | | | (SEQ ID NO: 5) |
| BC6 | 1 | | Homo sapiens mRNA; | AL080059 |
| 89.5% | | | cDNA DKFZp564H142 | (SEQ ID: 1) |
| 87.2% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | HS1119D91 | Similar to S68401 (cattle) | NM_012261 |
| | | | glucose induced gene | (SEQ ID NO: 12) |
| | 4 | GCN1L1 | GCN1 (general control of | N38891 |
| | | | amino-acid synthesis 1, | (SEQ ID NO: 4) |
| | | | yeast)-like 1 | |
| | 5 | EXT1 | exostoses (multiple) 1 | NM_000127 |
| | | | | (SEQ ID NO: 5) |
| BC7 | 1 | | Homo sapiens mRNA; | AL080059 |
| 89.5% | | | cDNA DKFZp564H142 | (SEQ ID: 1) |
| 87.2% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | GCN1L1 | GCN1 (general control of | N38891 |
| | | | amino-acid synthesis 1, | (SEQ ID NO: 4) |
| | | | yeast)-like 1 | |
| | 4 | EXT1 | exostoses (multiple) 1 | NM_000127 |
| | | | | (SEQ ID NO: 5) |
| | 5 | | Homo sapiens cDNA: | AK026372 |
| | | | FLJ22719 fis, clone | (SEQ ID NO: 10) |
| | | | HSI14307 | |
| BC8 | 1 | | Homo sapiens mRNA; | AL080059 |
| 94.7% | | | cDNA DKFZp564H142 | (SEQ ID: 1) |
| 89.7% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | WISP1 | WNT1 inducible signaling | NM_003882 |
| | | | pathway protein 1 | (SEQ ID NO: 13) |
| | 4 | GCN1L1 | GCN1 (general control of | N38891 |
| | | | amino-acid synthesis 1, | (SEQ ID NO: 4) |
| | | | yeast)-like 1 | |
| | 5 | EXT1 | exostoses (multiple) 1 | NM_000127 |
| | | | | (SEQ ID NO: 5) |
| BC9 | 1 | | Homo sapiens mRNA; | AL080059 |
| 89.5% | | | cDNA DKFZp564H142 | (SEQ ID: 1) |
| 83.3% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | FGF18 | fibroblast growth factor 18 | NM_003862 |
| | | | | (SEQ ID NO: 14) |
| | 4 | GCN1L1 | GCN1 (general control of | N38891 |
| | | | amino-acid synthesis 1, | (SEQ ID NO: 4) |
| | | | yeast)-like 1 | |
| | 5 | EXT1 | exostoses (multiple) 1 | NM_000127 |
| | | | | (SEQ ID NO: 5) |
| BC10 | 1 | | Homo sapiens mRNA; | AL080059 |
| 89.5% | | | cDNA DKFZp564H142 | (SEQ ID: 1) |
| 87.2% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | GCN1L1 | GCN1 (general control of | N38891 |
| | | | amino-acid synthesis 1, | (SEQ ID NO: 4) |
| | | | yeast)-like 1 | |
| | 4 | EXT1 | exostoses (multiple) 1 | NM_000127 |
| | | | | (SEQ ID NO: 5) |
| | 5 | GSTM3 | Glutathione S-transferase | NM_000849 |
| | | | M3 (brain) | (SEQ ID NO: 15) |
| BC11 | 1 | | Homo sapiens mRNA; | AL080059 |
| 78.9% | | | cDNA DKFZp564H142 | (SEQ ID: 1) |
| 87.2% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | MCCC1 | 3-methylcrotonyl-CoA | NM_020166 |
| | | | carboxylase biotin- | (SEQ ID NO: 16) |
| | | | containing subunit | |
| | 4 | FGF18 | fibroblast growth factor 18 | NM_003862 |
| | | | | (SEQ ID NO: 14) |
| | 5 | | ESTs | AA555029 |
| | | | | (SEQ ID NO: 17) |
| BC12 | 1 | | Homo sapiens mRNA; | AL080059 |
| 94.7% | | | cDNA DKFZp564H142 | (SEQ ID: 1) |
| 85.9% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | GCN1L1 | GCN1 (general control of | N38891 |
| | | | amino-acid synthesis 1, | (SEQ ID NO: 4) |
| | | | yeast)-like 1 | |
| | 4 | IP6K2 | mammalian inositol | AL137514 |
| | | | hexakisphosphate kinase 2 | (SEQ ID NO: 18) |
| | 5 | EXT1 | exostoses (multiple) 1 | NM_000127 |
| | | | | (SEQ ID NO: 5) |
| BC13 | 1 | | Homo sapiens mRNA; | AL080059 |
| 84.2% | | | cDNA DKFZp564H142 | (SEQ ID: 1) |
| 87.2% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | GCN1L1 | GCN1 (general control of | N38891 |
| | | | amino-acid synthesis 1, | (SEQ ID NO: 4) |
| | | | yeast)-like 1 | |
| | 4 | EXT1 | exostoses (multiple) 1 | NM_000127 |
| | | | | (SEQ ID NO: 5) |
| | 5 | CA9 | carbonic anhydrase IX | NM_001216 |
| | | | | (SEQ ID NO: 19) |
| BC14 | 1 | | Homo sapiens mRNA; | AL080059 |
| 94.7% | | | cDNA DKFZp564H142 | (SEQ ID: 1) |
| 84.6% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | MCCC1 | 3-methylcrotonyl-CoA | NM_020166 |
| | | | carboxylase biotin- | (SEQ ID NO: 16) |
| | | | containing subunit | |
| | 4 | GCN1L1 | GCN1 (general control of | N38891 |
| | | | amino-acid synthesis 1, | (SEQ ID NO: 4) |
| | | | yeast)-like 1 | |
| | 5 | EXT1 | exostoses (multiple) 1 | NM_000127 |
| | | | | (SEQ ID NO: 5) |
| BC15 | 1 | | Homo sapiens mRNA; | AL080059 |
| 89.5% | | | cDNA DKFZp564H142 | (SEQ ID: 1) |
| 87.1% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | DKFZP76 | hypothetical protein | AB033043 |
| | | 1L0424 | DKFZp761L0424 | (SEQ ID NO: 20) |
| | 4 | HRASLS | H-REV107 protein-related | NM_020386 |
| | | | protein | (SEQ ID NO: 21) |
| | 5 | MMP9 | matrix metalloproteinase 9 | NM_004994 |
| | | | (gelatinase B, 92 kD | (SEQ ID NO: 22) |
| | | | gelatinase, 92 kD type IV | |
| | | | collagenase) | |
| BC16 | 1 | | Homo sapiens mRNA; | AL080059 |
| 89.5% | | | cDNA DKFZp564H142 | (SEQ ID: 1) |
| 87.1% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | ALDH4A1 | aldehyde dehydrogenase 4 | NM_003748 |
| | | | (glutamate gamma- | (SEQ ID NO: 8) |
| | | | semialdehyde | |
| | | | dehydrogenase; pyrroline-5- | |
| | | | carboxylate dehydrogenase) | |
| | 4 | GCN1L1 | GCN1 (general control of | N38891 |
| | | | amino-acid synthesis 1, | (SEQ ID NO: 4) |
| | | | yeast)-like 1 | |
| | 5 | EXT1 | exostoses (multiple) 1 | NM_000127 |
| | | | | (SEQ ID NO: 5) |
| BC17 | 1 | | Homo sapiens mRNA; | AL080059 |
| 94.7% | | | cDNA DKFZp564H142 | (SEQ ID: 1) |
| 87.1% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | GCN1L1 | GCN1 (general control of | N38891 |
| | | | amino-acid synthesis 1, | (SEQ ID NO: 4) |
| | | | yeast)-like 1 | |
| | 4 | EXT1 | exostoses (multiple) 1 | NM_000127 |
| | | | | (SEQ ID NO: 5) |
| | 5 | HSA250839 | gene for serine/threonine | NM_018401 |
| | | | protein kinase | (SEQ ID NO: 23) |
| BC18 | 1 | | Homo sapiens mRNA; | AL080059 |
| 73.7% | | | cDNA DKFZp564H142 | (SEQ ID: 1) |
| 84.6% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | | ESTs | AA834945 |
| | | | | (SEQ ID NO: 24) |
| | 4 | KIAA1442 | KIAA1442 protein | AB037863 |
| | | | | (SEQ ID NO: 25) |
| | 5 | EXT1 | exostoses (multiple) 1 | NM_000127 |
| | | | | (SEQ ID NO: 5) |
| BC19 | 1 | | Homo sapiens mRNA; | AL080059 |
| 89.5% | | | cDNA DKFZp564H142 | (SEQ ID: 1) |
| 88.4% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | GCN1L1 | GCN1 (general control of | N38891 |
| | | | amino-acid synthesis 1, | (SEQ ID NO: 4) |
| | | | yeast)-like 1 | |
| | 4 | EXT1 | exostoses (multiple) 1 | NM_000127 |
| | | | | (SEQ ID NO: 5) |
| | 5 | ESTs | | AA828380 |
| | | | | (SEQ ID NO: 26) |
| BC20 | 1 | | Homo sapiens mRNA; | AL080059 |
| 87.2% | | | cDNA DKFZp564H142 | (SEQ ID: 1) |
| 89.5% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | | ESTs | AL310524 |
| | | | | (SEQ ID NO: 11) |
| | 4 | GCN1L1 | GCN1 (general control of | N38891 |
| | | | amino-acid synthesis 1, | (SEQ ID NO: 4) |
| | | | yeast)-like 1 | |
| | 5 | | Homo sapiens cDNA: | AK026372 |
| | | | FLJ22719 fis, clone | (SEQ ID NO: 10) |
| | | | HSI14307 | |
| BC21 | 1 | | Homo sapiens mRNA; | AL080059 |
| 88.5% | | | cDNA DKFZp564H142 | (SEQ ID: 1) |
| 89.5% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | MGAT4A | mannosyl (alpha-1,3-)- | NM_012214 |
| | | | glycoprotein beta-1,4-N- | (SEQ ID NO: 27) |
| | | | acetylglucosaminyltransferase, | |
| | | | isoenzyme A | |
| | 4 | GCN1L1 | GCN1 (general control of | N38891 |
| | | | amino-acid synthesis 1, | (SEQ ID NO: 4) |
| | | | yeast)-like 1 | |
| | 5 | EXT1 | exostoses (multiple) 1 | NM_000127 |
| | | | | (SEQ ID NO: 5) |
| BC22 | 1 | | Homo sapiens mRNA; | AL080059 |
| 88.5% | | | cDNA DKFZp564H142 | (SEQ ID: 1) |
| 89.5% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | MGC2827 | ESTs | NM_023940 |
| | | | | (SEQ ID NO: 28) |
| | 4 | GCN1L1 | GCN1 (general control of | N38891 |
| | | | amino-acid synthesis 1, | (SEQ ID NO: 4) |
| | | | yeast)-like 1 | |
| | 5 | EXT1 | exostoses (multiple) 1 | NM_000127 |
| | | | | (SEQ ID NO: 5) |
| BC23 | 1 | | Homo sapiens mRNA; | AL080059 |
| 91.0% | | | cDNA DKFZp564H142 | (SEQ ID: 1) |
| 78.9% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | | ESTs | AL310524 |
| | | | | (SEQ ID NO: 11) |
| | 4 | FGF18 | fibroblast growth factor 18 | NM_003862 |
| | | | | (SEQ ID NO: 14) |
| | 5 | | Homo sapiens cDNA: | AK026372 |
| | | | FLJ22719 fis, clone | (SEQ ID NO: 10) |
| | | | HSI14307 | |
| BC24 | 1 | | Homo sapiens mRNA; | AL080059 |
| 89.5% | | | cDNA DKFZp564H142 | (SEQ ID: 1) |
| 87.1% | | | (from clone | |
| | | | DKFZp564H142) | |
| | 2 | FLJ21924 | Homo sapiens cDNA: | NM_024774 |
| | | | FLJ21924 fis, clone | (SEQ ID N0; 2) |
| | | | HEP04086 | |
| | 3 | MCCC1 | 3-methylcrotonyl-CoA | NM_020166 |
| | | | carboxylase biotin- | (SEQ ID NO: 16) |
| | | | containing subunit | |
| | 4 | GCN1L1 | GCN1 (general control of | N38891 |
| | | | amino-acid synthesis 1, | (SEQ ID NO: 4) |
| | | | yeast)-like 1 | |
| | 5 | | ESTs | AW024884 |
| | | | | (SEQ ID N0: 29) |
| BC25 | 1 | | Homo sapiens mRNA; | AL080059 |
| 84.2% | | | cDNA DKFZp564H142 | (SEQ ID: 1) |
| 91.0% | | | (from clone | |
| | | | DKFZp564H142) | |
| | 2 | FLJ21924 | Homo sapiens cDNA: | NM_024774 |
| | | | FLJ21924 fis, clone | (SEQ ID NO; 2) |
| | | | HEP04086 | |
| | 3 | | ESTs | AL310524 |
| | | | | (SEQ ID NO: 11) |
| | 4 | WISP1 | WNT1 inducible signaling | NM_003882 |
| | | | pathway protein 1 | (SEQ ID NO: 13) |
| | 5 | | Homo sapiens cDNA: | AK026372 |
| | | | FLJ22719 fis, clone | (SEQ ID NO: 10) |
| | | | HSI14307 | |

1. A composition comprising a breast cancer biomarker consisting of between 3 and 73 different probe sets, wherein at least 40% of the different probe sets comprise one or more isolated polynucleotides that selectively hybridize to a nucleic acid according to one of SEQ ID NO:1-29 or complements thereof; wherein the different probe sets in total selectively hybridize to at least three of the recited nucleic acids according to SEQ ID NO:1-29 or complements thereof.

2. The composition of claim 1 wherein the different polynucleotide probe sets in total selectively hybridize to at least five of the recited nucleic acids according to SEQ ID NO:1-29 or complements thereof.

3. The composition of claim 1 wherein at least 50% of the different probe sets comprise one or more isolated polynucleotides that selectively hybridize to a nucleic acid according to one of SEQ ID NO:1-29 or complements thereof.

4. The composition of claim 1 wherein the different probe sets in total selectively hybridize to at least 3 of the following:
(a) SEQ ID NO:1, or its complement;
(b) SEQ ID NO:2, or its complement;
(c) SEQ ID NO:4, or its complement; and
(d) SEQ ID NO:5, or its complement.

5. A method for classifying a breast tumor comprising:
(a) contacting a mRNA-derived nucleic acid sample obtained from a subject having a breast tumor with nucleic acid probes that, in total, selectively hybridize to three or more nucleic acid targets selected from the group consisting of SEQ ID NO:1-29 or complements thereof; wherein the contacting occurs under conditions to promote selective hybridization of the nucleic acid probes to the nucleic acid targets, or complements thereof, present in the nucleic acid sample;
(b) detecting formation of hybridization complexes between the nucleic acid probes to the nucleic acid targets, or complements thereof, wherein a number of such hybridization complexes provides a measure of gene expression of the one or more nucleic acids according to SEQ ID NO:1-29; and
(c) correlating an alteration in gene expression of the one or more nucleic acids according to SEQ ID NO:1-29 relative to control with a risk of breast cancer recurrence.
